(19) United States
(12) Patent Application Publication
(10) Pub. No.: US 2026/0240882 A1
(43) Pub. Date: Aug. 20, 2026

(54) USE OF INHIBITORS OF HORMONE-SENSITIVE LIPASE (HSL) FOR THE TREATMENT OF DIFFUSE GLIOMAS

(71) Applicants: INSTITUT NATIONAL DE LA SANTÉ ET DE LA RECHERCHE MÉDICALE, Paris (FR); UNIVERSITE DE BORDEAUX, Bordeaux (FR)

(72) Inventors: Lucie BRISSON, Pessac Cedex (FR); Cloé TESSIER, Pessac Cedex (FR)

(21) Appl. No.: 19/330,284

(22) Filed: Sep. 16, 2025

Related U.S. Application Data
(60) Provisional application No. 63/696,552, filed on Sep. 19, 2024.

(51) Int. Cl. A61K 31/69; A61K 31/495; A61K 31/5375; A61K 31/7105; A61P 35/00

(52) U.S. Cl. CPC A61K 31/69; A61K 31/495; A61K 31/5375; A61K 31/7105; A61P 35/00

(57) ABSTRACT

Diffuse gliomas and in particular glioblastoma (GB) tumors exhibit heterogeneity and adapt to their environment via lipid droplet (LD) catabolism, which aids in cancer metabolism and signaling. Using 3D in-vitro and in-vivo models, the inventors observed that inhibiting hormone-sensitive lipase (HSL) leads to LD accumulation, reducing GB cell proliferation and inducing apoptosis. This suggests that LD degradation supports GB growth by releasing free fatty acids and cholesterol for membrane components and energy. The inventors showed that HSL inhibition decreases mitochondrial membrane potential and oxygen consumption, highlighting its role in GB aggressiveness and as a therapeutic target.

### RELATED APPLICATIONS

This application claims benefit of U.S. provisional application 63/696,552 filed on Sep. 19, 2024, which is incorporated into this application in its entirety.

### FIELD OF THE INVENTION

The present invention is in the field of medicine, in particular oncology.

### BACKGROUND OF THE INVENTION

Diffuse glioma is a term used to encompass a variety of poorly marginated infiltrating tumors of the central nervous system, which histologically appear similar to glial cells, specifically astrocytes and oligodendrocytes. These range in biological behavior from very indolent to extremely aggressive and this is reflected in grading that ranges from grade 1 to grade 4. In particular, glioblastoma (GB) represents the most aggressive form of primary brain tumors. This malignancy is notorious for its intratumor heterogeneity and the high plasticity of GB cells, which confer an extraordinary ability to adapt and survive under various microenvironmental pressures. A key player in these adaptive mechanisms is the lipid droplet (LD), an organelle crucial for maintaining lipid homeostasis by mediating lipid storage and release. LDs play a significant role in cancer metabolism and signaling. The role of metabolic adaptation, particularly through LD catabolism, has been identified as a major factor in the aggressiveness of GB. Understanding how GB cells exploit these metabolic pathways to thrive could unveil new therapeutic targets aimed at mitigating the tumor's invasive potential and improving patient outcomes.

### SUMMARY OF THE INVENTION

The present invention is defined by the claims. In particular, the present invention relates to use of inhibitors of hormone-sensitive lipase (HSL) for the treatment of diffuse gliomas.

### DETAILED DESCRIPTION OF THE INVENTION

The present invention relates to a method of treating a diffuse glioma in a patient in need thereof comprising administering to the patient a therapeutically effective amount of a HSL inhibitor.

As used herein, the term “patient” is interchangeable with the term “individual” or “subject”, and may refer to a patient to be treated by the methods disclosed herein. In particular, the patient suffers from a diffuse glioma. In some embodiments, the patient is a human infant. In some embodiments, the patient is a human child. In some embodiments, the patient is a human adult. In some embodiments, the patient is an elderly.

As used herein, the term “diffuse glioma” refers to a type of brain tumor characterized by its infiltrative nature, meaning the tumor cells spread widely and obscurely through the brain tissue. This category includes both pediatric-type diffuse gliomas and adult-type diffuse gliomas, each with distinct molecular and genetic profiles that influence their diagnosis, treatment, and prognosis.

In some embodiments, the method of the present invention is particularly suitable for the treatment of adult-type diffuse gliomas. As used herein, the term “adult-type diffuse glioma” refers to a category of diffuse gliomas that are typically diagnosed in adults. This category includes three main subtypes: Astrocytoma (IDH-mutant), Oligodendroglioma (IDH-mutant and 1p/19q-codeleted), and Glioblastoma (IDH-wildtype). These subtypes are distinguished based on their molecular and genetic characteristics, which have significant implications for prognosis and treatment strategies.

In some embodiments, the method of the present invention is particularly suitable for the the treatment of glioblastoma. As used herein, the term “glioblastoma” has its general meaning in the art and represents the most aggressive and most common type of cancer that originates in the brain, and has a very poor prognosis for survival. Only tumors that are IDH wild type are now classified as glioblastoma (Louis, David N., et al. “The 2021 WHO classification of tumors of the central nervous system: a summary.” Neuro-oncology 23.8 (2021): 1231-1251).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “hormone-sensitive lipase” or “HSL” refers to an enzyme that plays a key role in the hydrolysis of stored triglycerides into free fatty acids and glycerol. More particularly, HSL is an intracellular neutral lipase that mediates the hydrolysis of broad substrates such as tri-, di-, and monoacylglycerol (TG, DG and MG), cholesterylester, retinyl ester and water soluble ester substrates.

As used herein, the term “inhibitor” refers to a compound that decreases the magnitude of at least one activity, signaling or expression of a molecule compared to the magnitude of the activity, signaling or expression observed in the absence of the inhibitor. In some instances, an inhibitor will substantially decrease the magnitude of at least one activity, signaling or expression of a molecule compared to the magnitude of the activity or expression observed in the absence of the inhibitor. In some instances, an inhibitor will completely diminish the magnitude of at least one activity, signaling or expression of a molecule compared to the magnitude of the activity, signaling or expression observed in the absence of the inhibitor. As used herein, the term “HSL inhibitor” refers to a compound that can inhibit the activity and/or expression of hormone-sensitive lipase (HSL). Without wishing to be bound by a theory, a HSL inhibitor can act by a number of different pathways. The HSL inhibitors can encompass numerous classes of chemical molecules, e.g., small organic or inorganic molecules, polysaccharides, biological macromolecules, e.g., peptides, proteins, peptide analogs and derivatives, peptidomimetics, antibodies, antibody fragments, nucleic acids, nucleic acid analogs and derivatives, an extract made from biological materials such as bacteria, plants, fungi, or animal cells or tissues, naturally occurring or synthetic compositions. Thus, a HSL inhibitor can be a molecule of any type that interferes with the activity or expression of HSL, for example, either by decreasing transcription or translation of HSL encoding nucleic acid, or by inhibiting or blocking HSL activity, or both. Examples of HSL inhibitors include, but are not limited to, antisense polynucleotides, interfering RNAs, catalytic RNAs, RNA-DNA chimeras, HSL-specific aptamers, anti-HSL antibodies, HSL-binding fragments of anti-HSL antibodies, HSL-binding small molecules, HSL-binding peptides, and other polypeptides that specifically bind HSL (including, but not limited to, HSL-binding fragments of one or more HSL ligands, optionally fused to one or more additional domains), such that the interaction between the HSL inhibitor and HSL results in a reduction or cessation of HSL activity or expression. It will be appreciated that HSL inhibitors described herein may be strong inhibitors of HSL. In some embodiments, a HSL inhibitor inhibits the biological activity of the HSL receptor by at least 5%, at least 10%, at least 15%, at least 20%, at least 30%, at least 40%, at least 50%, at least 60%, at least 70%, at least 75%, at least 80%, at least 85%, at least 90%, or at least 95% relative to a control. In some embodiments, a HSL inhibitor completely abrogates the biological activity of the HSL receptor relative to a control. A control can comprise a sample that is not treated with a HSL inhibitor.

In some embodiments, the HSL inhibitor is a small molecule. As used herein, the term “small molecule” refers to a low molecular weight organic compound, typically less than 900 daltons, which can regulate a biological process, with a size small enough to rapidly diffuse across cell membranes and reach intracellular sites of action. Small molecules can be designed to interact with specific proteins, nucleic acids, or other biomolecules to modulate their activity. Small molecules that act as HSL inhibitors are well known in the art and include those described in:
Ogiyama T, Yamaguchi M, Kurikawa N, Honzumi S, Terayama K, Nagaoka N, Yamamoto Y, Kimura T, Sugiyama D, Inoue S I. Design, synthesis, and pharmacological evaluation of a novel series of hormone sensitive lipase inhibitor. Bioorg Med Chem. 2017 Sep. 1; 25(17):4817-4828. doi: 10.1016/j.bmc.2017.07.028. Epub 2017 Jul. 17. PMID: 28756012.
Ogiyama T, Yamaguchi M, Kurikawa N, Honzumi S, Yamamoto Y, Sugiyama D, Takakusa H, Inoue S I. Identification of a novel hormone sensitive lipase inhibitor with a reduced potential of reactive metabolites formation. Bioorg Med Chem. 2017 Apr. 1; 25(7):2234-2243. doi: 10.1016/j.bmc.2017.02.045. Epub 2017 Feb. 27. PMID: 28279560.
Ogiyama T, Yamaguchi M, Kurikawa N, Honzumi S, Yamamoto Y, Sugiyama D, Inoue S. Identification of a novel boronic acid as a potent, selective, and orally active hormone sensitive lipase inhibitor. Bioorg Med Chem. 2016 Aug. 15; 24(16):3801-7. doi: 10.1016/j.bmc.2016.06.022. Epub 2016 Jun. 11. PMID: 27338659.
Ebdrup S, Refsgaard H H, Fledelius C, Jacobsen P. Synthesis and structure-activity relationship for a novel class of potent and selective carbamate-based inhibitors of hormone selective lipase with acute in vivo antilipolytic effects. J Med Chem. 2007 Nov. 1; 50(22):5449-56. doi: 10.1021/jm0607653. Epub 2007 Oct. 5. PMID: 17918819.
de Jong J C, Sprensen L G, Tornqvist H, Jacobsen P. Carbazates as potent inhibitors of hormone-sensitive lipase. Bioorg Med Chem Lett. 2004 Apr. 5; 14(7):1741-4. doi: 10.1016/j.bmcl.2004.01.038. PMID: 15026062.

In some embodiments, the HSL inhibitor of the present invention is HSL-IN-1 (also known as compound 24b) (CAS No.: 2095156-13-9) as described in Ogiyama, Tomoko, et al. “Identification of a novel hormone sensitive lipase inhibitor with a reduced potential of reactive metabolites formation.” Bioorganic & medicinal chemistry 25.7 (2017): 2234-2243 and having the formula of:

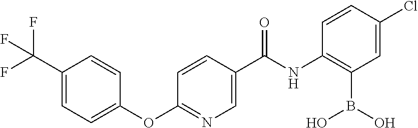 [SMILES: O=C(Nc1ccc(Cl)cc1B(O)O)c1ccc(Oc2ccc(C(F)(F)F)cc2)nc1]

In some embodiments, the HSL inhibitor of the present invention is 4-chlorophenyl)N-morpholin-4-ylcarbamate (also known as Hi 76-0079 or NNC0076-0079) having the formula of:

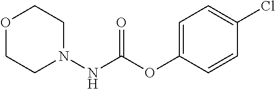 [SMILES: O=C(NN1CCOCC1)Oc1ccc(Cl)cc1]

In some embodiments, a HSL inhibitor is a nucleic acid. Exemplary HSL nucleic acid inhibitors include, but are not limited to, antisense oligonucleotides, siRNAs, shRNAs, microRNAs, aptamers, ribozymes and decoy oligonucleotides. A HSL nucleic acid inhibitor can inhibit the expression of a HSL gene.

In some embodiments, the HSL inhibitor of the present invention is administered to the patient in combination with chemotherapy.

As used herein, the term “chemotherapy” refers to any therapy that consists in administering a chemical agent with therapeutic usefulness in the treatment of diffuse gliomas, in particular glioblastoma. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these drugs are directly toxic to cancer cells and do not require immune stimulation. Suitable chemotherapeutic agents are described, for example, in Slapak and Kufe, Principles of Cancer Therapy, Chapter 86 in Harrison's Principles of Internal medicine, 14th edition; Perry et at, Chemotherapeutic, Ch 17 in Abeloff, Clinical Oncology 2nd ed., 2000 ChrchillLivingstone, Inc.; Baltzer L. and Berkery R. (eds): Oncology Pocket Guide to Chemotherapeutic, 2nd ed. St. Louis, mosby-Year Book, 1995; Fischer D. S., Knobf M. F., Durivage H J. (eds): The Cancer Chemotherapeutic Handbook, 4th ed. St. Louis, Mosby-Year Handbook.

In some embodiments, the chemotherapeutic agent is selected an alkylating agent. Selected alkylating agents include: altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, and trabectedin.

In some embodiments, the chemotherapeutic agent is temozolomide (TMZ). As used herein, “temozolomide” refers to a triazine alkylating agent and more specifically compound of formula 3,4-Dihydro-3-methyl-4-oxoimidazo[5,1-d][1,2,3,5]tetrazine-8-carboxamide and pharmaceutically acceptable salts thereof (CAS number of 85622-93-1).

In some embodiments, the HSL inhibitor of the present invention is administered to the patient in combination with radiotherapy.

As used herein, the term “radiotherapy” is used for the treatment of diseases of oncological nature with irradiation corresponding to ionizing radiation. Ionizing radiation deposits energy that injures or destroys cells in the area being treated (the target tissue) by damaging their genetic material, making it impossible for these cells to continue to grow. In some embodiments, the radiotherapy is a whole-brain radiotherapy (WBRT).

In some embodiment, the radiotherapy consists in exposing the tumor to be treated to an efficient dose of ionizing radiations. Typically gamma rays are used. Gamma rays are produced spontaneously as certain elements (such as radium, uranium, and cobalt 60) release radiation as they decompose, or decay. A person of ordinary skill in the radiotherapy art knows how to determine an appropriate dosing and application schedule, depending on the nature of the disease and the constitution of the patient. In particular, the person knows how to assess dose-limiting toxicity (DLT) and how to determine the maximum tolerated dose (MTD) accordingly. Typically, the amount of radiation varies depending on the type and stage of cancer being treated. For curative cases, the typical total dose for a solid tumor ranges from 20 to 120 Gy. In particular, the patient is administered with the standard of care to treat patients with glioblastoma. Such clinical standard comprises subjecting the subject to a dose of 2 Gy/day for 5 days, followed by 2 days of rest, for 6 consecutive weeks with a total dose of 60 Gy.

In some embodiments, the HSL inhibitor of the present invention is administered to the patient in combination with Tumor Treating Fields therapy. As used herein, the term “Tumor Treating Fields therapy” refers to a type of cancer treatment that uses alternating electric fields to disrupt the division of cancer cells. This non-invasive therapy targets dividing cells by applying low-intensity, intermediate-frequency electric fields to the tumor site, thereby impairing cell division and potentially leading to cell death. The therapy is typically administered using a device that generates these electric fields, which are delivered through electrodes placed on the patient's skin around the area of the tumor. Tumor Treating Fields therapy has shown efficacy in treating various types of cancer, particularly glioblastoma and other aggressive brain tumors.

As used herein, the term “combination” is intended to refer to all forms of administration that provide a first therapy together with a further (second, third . . . ) therapy. The therapies may be administered simultaneously, separately or sequentially and in any order. Therapies administered in combination have biological activity in the subject to which the therapies are delivered. Within the context of the invention, a combination thus comprises at least 2 different therapies, and wherein the first therapy is chemotherapy, radiotherapy or Tumor Treating Fields therapy, the second therapy is a HSL inhibitor. In some instance, the combination of the present invention results in the synthetic lethality of the cancer cells. In some embodiments, the patient is first administered with at least one cycle (C1) of administering a HSL inhibitor followed by administration of at least one cycle (C2) of chemotherapy, radiotherapy or Tumor Treating Fields therapy. As used herein, the term “cycle” refers to a period of time during the treatment is administered to the patient. Typically, in cancer therapy a cycle of therapy is followed by a rest period during which no treatment is given. Following the rest period, one or more further cycles of therapy may be administered, each followed by additional rest periods. In some embodiments, cycle (C1) comprises administering a dose of a HSL inhibitor daily or every 2, 3, 4, or 5 days. In some embodiments, a HSL inhibitor is administered continuously (i.e. every day) during cycle (C1). In some embodiments the administration of a dose of a HSL inhibitor is alternated with the administration of a dose of chemotherapy, radiotherapy or Tumor Treating Fields therapy. Typically cycle (C1) can last one or more days, but is usually one, two, three or four weeks long. In some embodiments cycle (C1) is repeated at least 2, 3, 4, 5, 6, 7, 8, 9 or 10 times before administering cycle (C2).

As used herein, the expression “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the inhibitor of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: HSL inhibition leads to LD accumulation and impairs growth and survival of GB cells.

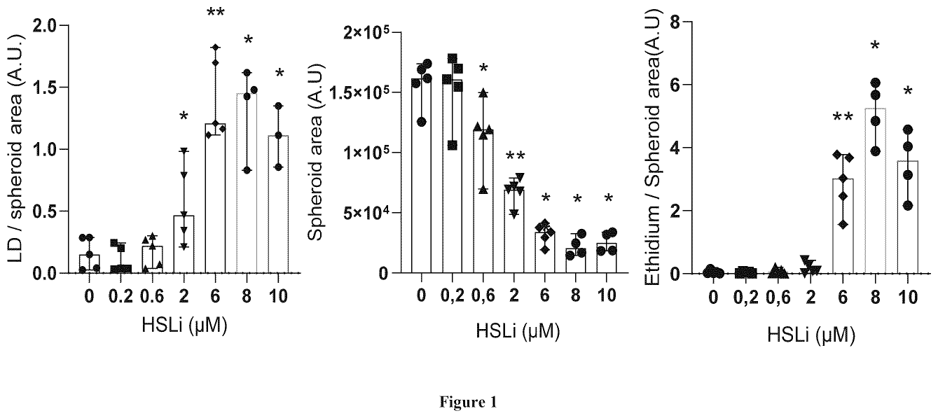

FIG. 2: HSL inhibition is sensitizing BTSC25 cells to irradiation and TMZ treatments.

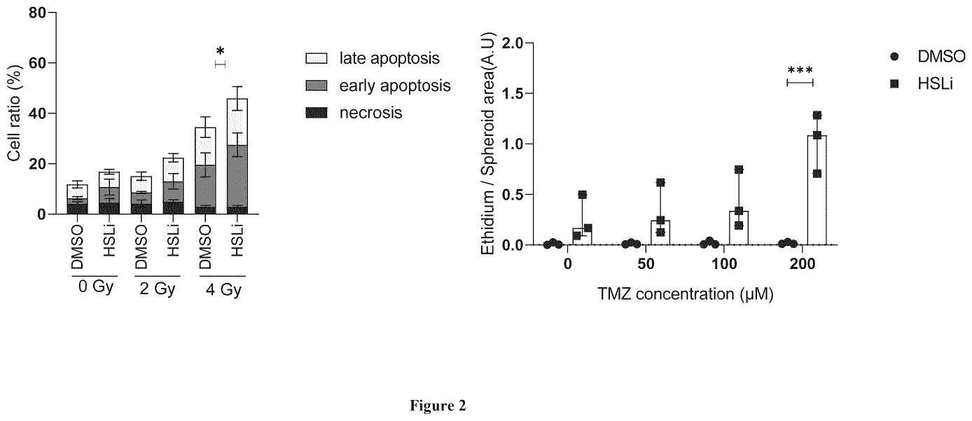

A. BTSC25 cells were treated with DMSO or HSLi (2 μM) and irradiated with 0, 2 or 4 Gy. After 7 days cells were analysed by flux cytometer after Annexine/Pi incubation. B. BTSC25 spheroid treated with DMSO or HSLi (2 μM) combined to a range of TMZ treatment for 7 days, dead cells were stained with ethidium homodimer.

FIG. 3: In vivo experiment with CTL or HSL-KO cells implanted in mouse brains. A. Bioluminescence following of the tumor growth of implanted BTSC25 CTL or HSL-KO cells. B. Survival curve of mice implanted either with the BTSC25 CTL cell line or HSL-KO cell line.

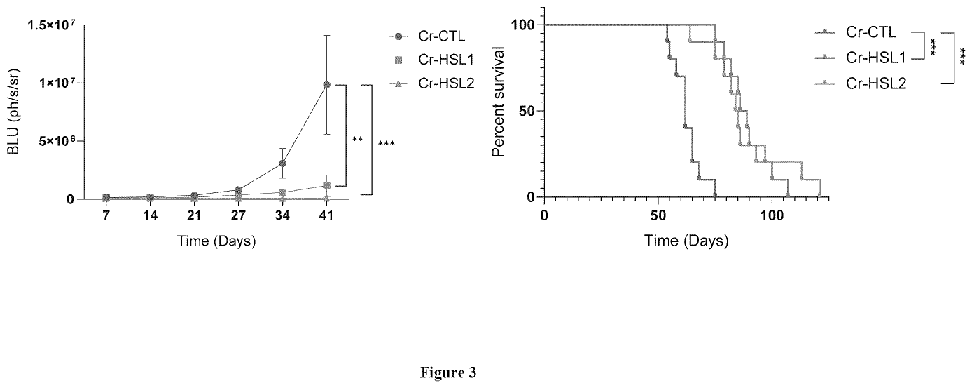

### EXAMPLE

Glioblastoma (GB), the most aggressive primary brain tumors, are characterized by intratumor heterogeneity and a high plasticity of GB cells enabling adaptation and survival under microenvironment pressure. Lipid droplets are key organelles at the interface between lipid storage and release to maintain lipid homeostasis and participating in cancer metabolism and signaling. We aim to understand the role of metabolic adaptation in GB aggressiveness, by investigating the role of lipid droplet (LD) catabolism.

Using 3D in-vitro models of GB and tumors implanted in-vivo in mice, we show a heterogeneous distribution of LD, suggesting that the dynamics of LD depends on the tumor microenvironment. To characterize LD degradation in GB and the role of this catabolic mechanism, we have inhibited the main processes of LD degradation; lipolysis and lipophagy, by targeting two cytosolic lipases: adipose-triglyceride lipase and hormone-sensitive lipase (HSL) and one lysosomal lipase: lysosomal acid lipase. We show that only the inhibition of HSL leads to a dose-dependent accumulation of LD which is associated with a decrease in GB cell proliferation and an induction of cell death by apoptosis.

We hypothesize that lipid droplet degradation can promote GB growth, by releasing free fatty acids and cholesterol that can be used as membrane components, signaling molecules or source of energy. We show that HSL inhibition induces a decrease in free fatty acid quantity with no modification of cholesterol and phospholipids. To understand the effect of HSL inhibition, we first investigate mitochondrial metabolism. Inhibition of HSL induces a decrease in mitochondrial membrane potential measured by TMRM, associated with a decrease in oxygen consumption by mitochondria. The effects of HSL on mitochondrial respiration has been confirmed with the genetic depletion of HSL by crispr-cas9. This metabolic plasticity may have a major role in GB intra-tumor heterogeneity and cancer progression.

In conclusion, these results highlight the role of LD degradation in GB cell aggressiveness and the link with mitochondrial metabolism. This study could lead to the proposition of HSL as a new target in GB.

### REFERENCES

Throughout this application, various references describe the state of the art to which this invention pertains. The disclosures of these references are hereby incorporated by reference into the present disclosure.

1. A method of treating a diffuse glioma in a patient in need thereof comprising administering to the patient a therapeutically effective amount of a HSL inhibitor.

2. The method according to claim 1 wherein the diffuse glioma is a pediatric-type diffuse glioma or adult-type diffuse glioma.

3. The method according to claim 2 wherein the adult-type diffuse glioma is glioblastoma.

4. The method according to claim 1 wherein the HSL inhibitor is administered to the patient in combination with chemotherapy, radiotherapy and/or Tumor Treating Fields therapy.

5. The method according to claim 1 wherein the HSL inhibitor is administered to the patient in combination with temozolomide.
